(19) United States
(12) Patent Application Publication
(10) Pub. No.: US 2010/0292777 A1
(43) Pub. Date: Nov. 18, 2010

(54) Stent

(75) Inventors: Michael P. Meyer, Richfield, MN (US); Samuel Robaina, Santa Rosa, CA (US); Dennis Boismier, Shorewood, MN (US); Jason T. Lenz, Maplewood, MN (US); Liza Davis, St. Michael, MN (US); Kim Robertson, Forest Lake, MN (US); Doug Thesingh, Minneapolis, MN (US); Cory Hitzman, St. Paul, MN (US); Timothy S. Girton, Edina, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 12/465,443

(22) Filed: May 13, 2009

(51) Int. Cl. A61F 2/06

(52) U.S. Cl. 623/1.16

(57) ABSTRACT

An expandable stent has a plurality of expandable rings formed of a plurality of struts and at least one first connector interconnecting adjacent expandable rings. The plurality of struts include a first strut and the at least one first connector has a first arm. A first portion of the first arm is engaged to a first portion of the first strut so that the first portion of the first arm and the first portion of the first strut define a through hole.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

Stents may be constructed and arranged to deliver a therapeutic agent. Examples of stents designed to deliver a therapeutic agent are discussed in U.S. Pat. No. 6,764,507 to Shanley, US Application Publication 2006/0122688 to Shanley, US Application Publication 2005/0261757, US Application Publication 2006/0229713 to Shanley and US Application Publication 2004/0220660 to Shanley, each of which are incorporated by reference in their entirety. FIGS. 1 and 2 are two examples of portions of prior art stent designs used to deliver a therapeutic agent. The stent 10 can be designed to deliver a therapeutic agent from wells/holes 26 located either only on the struts 12 or on both the struts 12 and connectors 22, as shown in FIGS. 1 and 2.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF TIE INVENTION

In at least one embodiment, the invention is directed to improved connector embodiments for a stent designed to deliver a therapeutic agent wherein some embodiments have improved flexibility, some embodiments improve the overall flexibility of the stent, some embodiments have improved strength, some embodiments have improved fatigue resistance and some embodiments have improved drug delivery.

These and other embodiments which characterize the invention are pointed out with particularity in the claims annexed hereto and forming a part hereof. However, for further understanding of the invention, its advantages and objectives obtained by its use, reference can be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a portion of a PRIOR ART stent design with wells.

FIG. 2 is a portion of another PRIOR ART stent design with wells.

FIG. 3 is a generic stent with connectors between adjacent columns of undulating bands being engaged at different locations along the strut.

FIG. 4 is a generic stent with an open cell design.

FIG. 5 is a view of a connector configuration for a drug delivery stent.

FIG. 6 is a view of a connector configuration for a drug delivery stent.

FIG. 7 is a view of a connector configuration for a drug delivery stent.

FIG. 8 is a view of a connector configuration for a drug delivery stent.

FIG. 9 is a view of a pair of connectors that alternate engagement at a peak and at mid-strut.

FIG. 10 is a view of a connector configuration that is zig-zag.

FIG. 11A is a view of a connector configuration that has zones of articulation, with the connector in an unflexed state.

FIG. 11B is a view of the connector of FIG. 11A in a flexed state.

FIG. 12 is the prior art stent of FIG. 1 with an alternate connector configuration.

FIG. 13 is the prior art stent of FIG. 1 with alternate connector configurations.

FIG. 14A is the prior art stent of FIG. 1 with an alternate connector configuration.

FIG. 14B is a side view of the alternate connector configuration of FIG. 14A.

FIG. 15 is the prior art stent of FIG. 1 with an alternate connector configuration.

FIG. 16 is the prior art stent of FIG. 1 with an alternate connector configuration.

FIG. 17 is the prior art stent of FIG. 1 with an alternate connector configuration.

FIG. 18 is the prior art stent of FIG. 2 with an alternate connector configuration.

FIG. 19 is the prior art stent of FIG. 2 with an alternate connector configuration.

FIG. 20 is the prior art stent of FIG. 2 with an alternate connector configuration.

FIG. 21 is the prior art stent of FIG. 2 with an alternate connector configuration.

FIG. 22 is the prior art stent of FIG. 2 with an alternate connector configuration.

FIG. 23 is the prior art stent of FIG. 2 with an alternate connector configuration.

FIG. 24 is the prior art stent of FIG. 2 with an alternate connector configuration.

FIG. 25 is a view of a generic stent with connectors that are coiled.

FIG. 26A is a view of a generic stent with a connector in a pre-expansion state.

FIG. 26B is the connector of FIG. 26A in a post-expansion state.

FIG. 27A is a view of a generic stent with a connector in a pre-expansion state.

FIG. 27B is the connector of FIG. 27A in a post-expansion state.

FIG. 28 is a view of a generic stent with a plurality of connectors.

FIG. 29 is a view of a generic stent with an undulating band of connectors engaging adjacent undulating bands of struts.

FIG. 30 is a side view of a stent tube made from two materials.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated. Thus, the following discussion about the different connectors 22 is intended to be illustrative and not exhaustive. The description will suggest many variations and alternatives to one of ordinary skill in the art. The various elements shown in the individual figures and described below may be combined or modified for combination as desired.

For the purposes of this disclosure, like reference numerals in the figures shall refer to like features unless otherwise indicated. The invention is directed to improved connector embodiments 22 for a stent 10 designed to deliver a therapeutic agent wherein some connector embodiments 22 have improved flexibility, some connector embodiments 22 improve the overall flexibility of the stent 10, some connector embodiments 22 have improved strength, and some connector embodiments 22 have improved fatigue resistance.

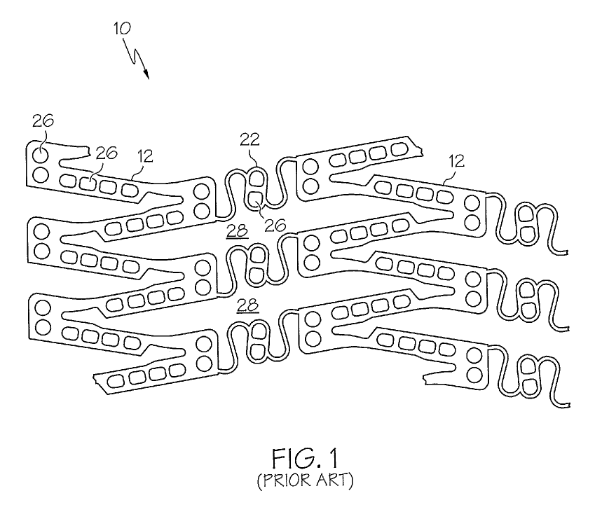

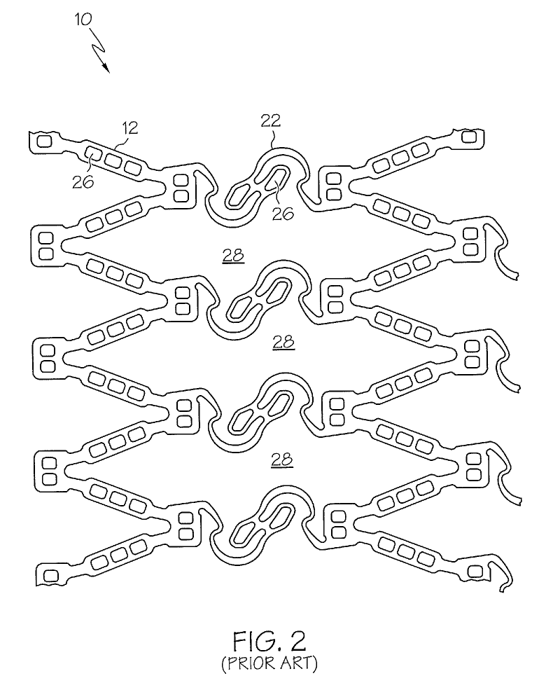

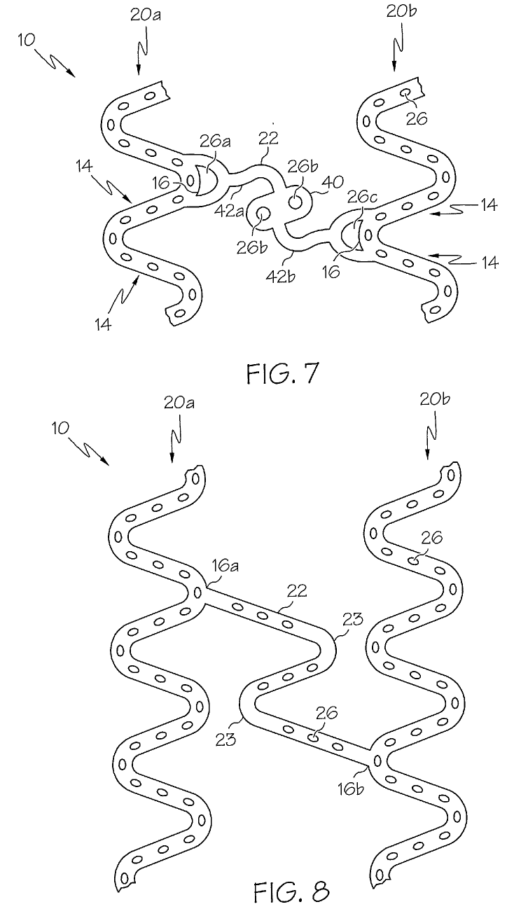

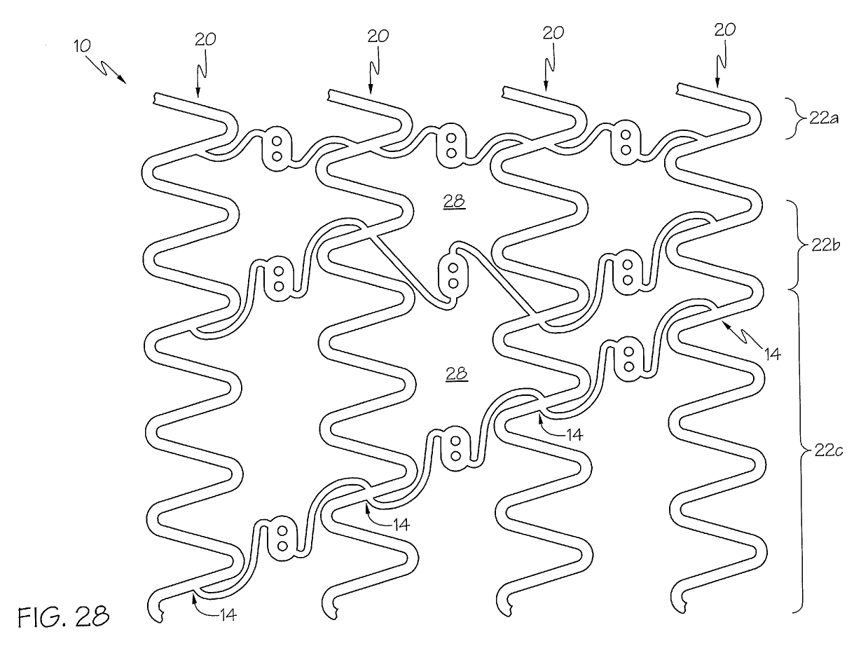

Each connector embodiment 22 can be used with any stent 10 design even though some figures show connector embodiments 22 engaging generic circumferential bands 20 of struts 12, while other figures show connector embodiments 22 with the stent 10 designs shown in FIGS. 1 and 2. Although the majority of the figures show an individual connector 22 between two portions of adjacent circumferential bands 20a,b, it is within the scope of the invention for a stent 10 to have a plurality of connectors 22 engaging a plurality of circumferential bands 20, as shown, for example, in FIG. 28. A stent 10 can have connectors 22 which each have the same configuration or connectors 22 that have a different configuration from other connectors 22. Furthermore, the connectors 22 can be arranged so that they are substantially longitudinal, as shown, for example, in FIG. 6 where the first and second ends of the connector 22 have substantially the same position about the circumference of the stent 10 (same circumferential position). Alternatively, the connectors 22 can be arranged so that they are circumferential, as shown, for example, in FIG. 7, where the first and second ends of the connector 22 have different positions about the circumference of the stent (circumferentially offset).

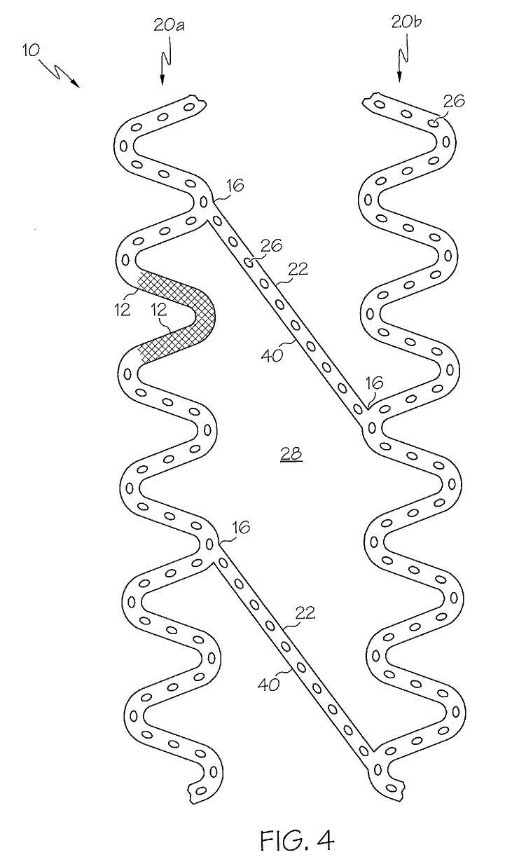

The first and/or second ends of circumferentially adjacent connectors 22 engaging adjacent circumferential bands 20a,b can be engaged to the same strut 12 pair, adjacent strut 12 pairs or separated by at least one strut 12 pair. As used in this application, a strut 12 pair is two circumferentially adjacent struts 12 engaged by a turn 23, as shown, for example, by cross-hatching in FIG. 4.

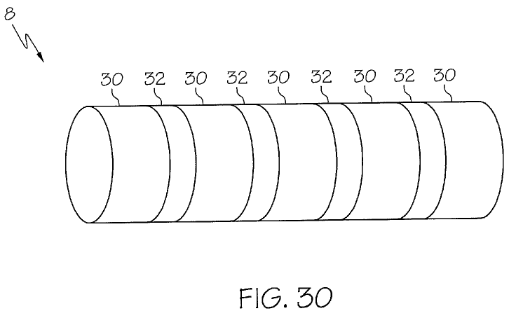

It is within the scope of the invention for the connector 22 to be made of any material. In some embodiments, the connector 22 is made of the same material as the circumferential bands 20 of the stent 10. In other embodiments, the connector 22 is made of different material than the circumferential bands 20 of the stent 10. Non-limiting examples of materials that can be used to make the circumferential bands 20 and/or the connectors 22 of the stent 10 are discussed in greater detail below. In at least one embodiment, the stent 10 is made from a stent tube 8 that has a plurality of first sections 30 and a plurality of second sections 32, as shown, for example, in FIG. 30. In this embodiment, the material forming the first sections 30 is different from the material forming the second sections 32 and the connectors 22 are formed in the second sections 32.

In at least one embodiment, the connector 22 is radially thicker than the circumferential bands 20. As used in this application, thickness is measured from a luminal side to an abluminal side of the stent 10. In some embodiments, additional material is added to the connector 22. Additional material can be added in any known manner, for example, but not limited to, vapor deposition, plating, injection molding, insert molding, press-fitting, spray coating, and ion implanting. In other embodiments, the stent 10 is made from a stent tube 8 that has a plurality of first sections 30 and a plurality of second sections 32, as shown, for example, in FIG. 30. In one embodiment, the first sections 30 are thinner than the second sections 32 and the connectors 22 are formed in the second sections 32. Thus, the connectors 22 are radially thicker than the circumferential bands 20, which are formed in the first sections 30.

In at least one embodiment, the at least one of the sections of the stent tube 8 forming the connectors 22 has a greater longitudinal length than the sections of the stent tube 8 forming the circumferential bands 20 of the stent 10.

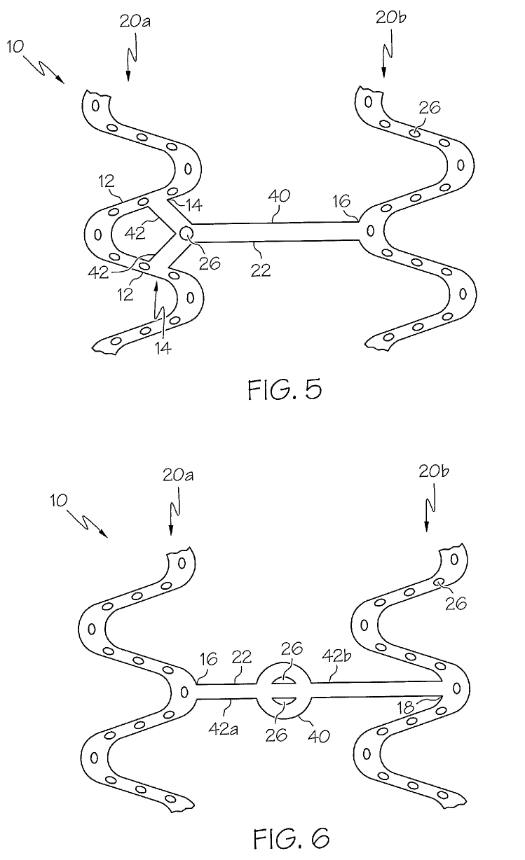

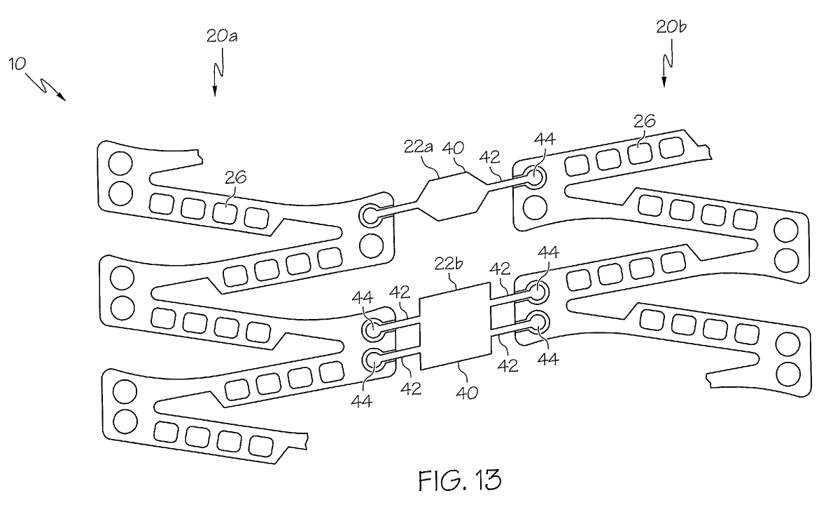

In at least one embodiment, the connector 22 has at least one body 40 and at least one arm 42, as shown, for example, in FIG. 5. It is within the scope of the invention for a connector 22 to have one, two, three, four, five, six, seven, eight, nine, ten or more bodies 40 and/or arms 42. It is within the scope of the invention for the body 40 to have any shape, for example, but not limited to round, oval, rectangular, square shaped, triangular, and polygonal. The body 40 can also have any orientation/ angle relative to the longitudinal axis of the stent 10. In at least one embodiment, the body 40 increases the surface area of the connector 22, as shown, for example, in FIG. 13.

In at least one embodiment, the body 40 increases the strength of the connector 22. In at least one embodiment, the strength of the connector 22 is increased by being processed by cold work. In some embodiments, the body 40 of the connector 22 undergoes a greater percentage of cold work than the circumferential bands 20. In other embodiments, the connectors 22 (body 40 and arms 42) undergo a greater percentage of cold work than the circumferential bands 20. In one embodiment, the stent tube 8 has a plurality of first sections 30 and a plurality of second sections 32, as shown, for example, in FIG. 30. The connectors 22 are formed in the plurality of second sections 32 which undergo cold work or a greater percentage of coldwork than first sections 30.

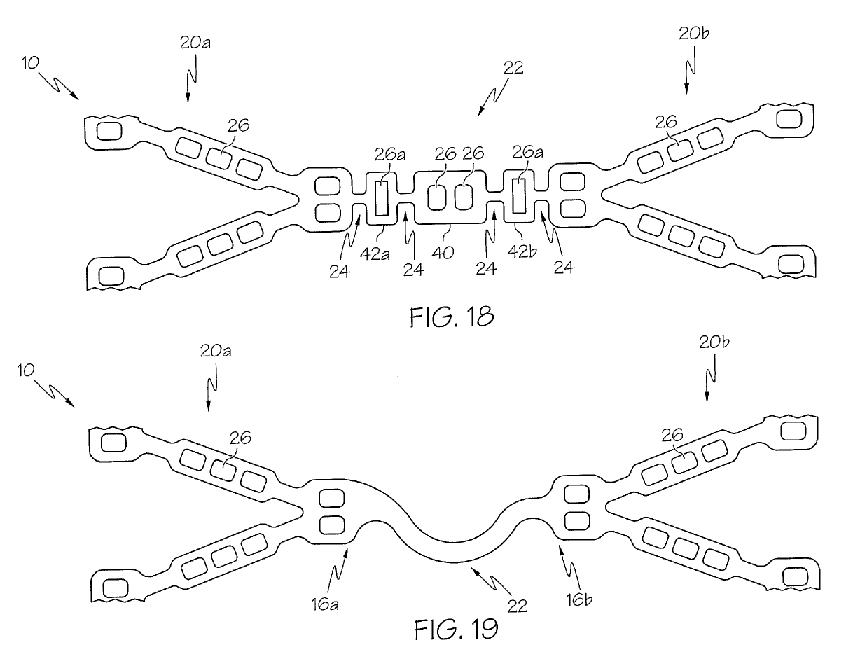

It is within the scope of the invention for the arms 42 to have any configuration, including, but not limited to, straight (shown, for example, in FIG. 6), curvilinear (shown, for example, by connector 22 in FIG. 19), zig-zag, O-shaped (shown, for example, in FIG. 18), V-shaped (shown, for example, in FIG. 16), U-shaped, X-shaped, Y-shaped (shown, for example, in FIG. 7), and any combination thereof. Thus an arm 42 can have the same configuration along its length or at least one portion of the arm 42 can have a different configuration than an adjacent portion of the arm 42. For example, the arms 42 of the connector 22 in FIG. 7 can be described as curvilinear Y-shaped arms 42, with one portion of the arm 42 being curvilinear and another portion of the arm 42 being Y-shaped. In some embodiments, the connector 22 has arms 42 that have the same configuration, as shown, for example, in FIG. 16. In other embodiments, the connector 22 has arms 42 that have different configurations. It is within the scope of the invention for the arms 42 to be engaged to any portion(s) of the body 40. It is also within the scope of the invention for the arms 42 to have any length. Thus, for example, if the connector 22 has two arms 42 engaged to a body 40, the arms 42 can be the same length or different lengths.

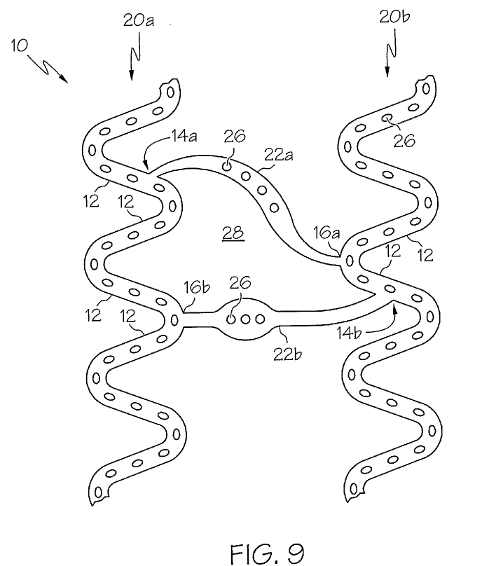

In at least one embodiment, the body 40 has a width at least equal to the width of the arm(s) 42. As used in this application, width is measured transverse to the length, from one side to another side of a strut 12 or connector 22, or a portion thereof. In some embodiments, the body 40 has the same width as the arms 42, as shown, for example, in FIG. 6. In other embodiments, the body 40 has substantially the same width as the arms 42, as shown, for example, in FIG. 5. As shown, for example, in FIG. 13, the body 40 has a greater width than the arms 42. In other embodiments, the connector 22 has clearly defined body 40 and arm 42 attached thereto, as shown, for example, by connector 22b of FIG. 9. In other embodiments, the connector 22 does not have a clearly defined body 40 and arm 42 attached thereto, as shown, for example, by connector 22a of FIG. 9 where the body 40 tapers into the arm 42 so that the start/end of the body 40 and the arm 42 is not clearly defined. In some embodiments, the connector 22 has a body 40 but no arms, as shown, for example, in FIG. 4.

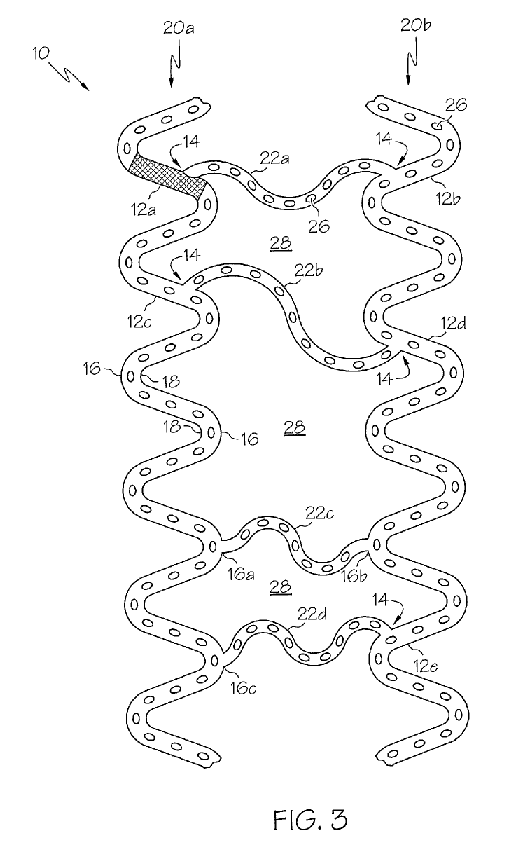

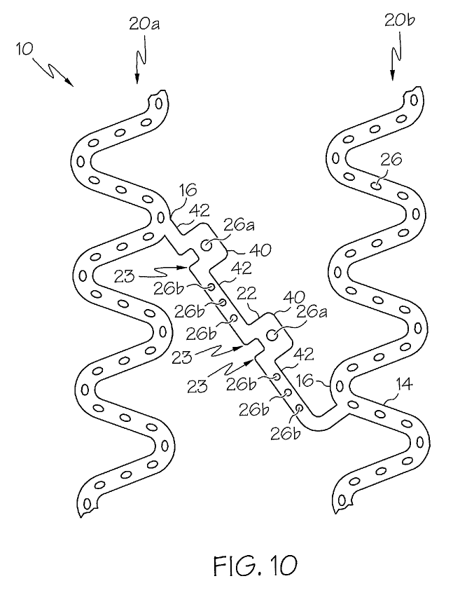

In at least one embodiment, the connector 22 defines at least one hole 26, as shown, for example, in FIG. 3. In some embodiments, a therapeutic agent is diposed within the hole 26, as discussed in greater detail below. It is within the scope of the invention for the connector 22 to have any number of holes 26, for example, but not limited to, one, two, three, four, five, six, seven, eight, nine, ten, eleven, twelve, thirteen, fourteen, fifteen, sixteen, seventeen, eighteen, nineteen, twenty, or more. In some embodiments, the at least one hole 26 is positioned in, and defined by, the body(ies) 40 of the connector 22, as shown, for example, in FIG. 6. In other embodiments, the at least one hole 26 is positioned in, and defined by, the arm(s) 42 of the connector 22, as shown, for example, in FIG. 10. In still other embodiments, the connector 22 has at least one hole 26 positioned in, and defined by, the body(ies) 40 of the connector 22 and at least one hole 26 positioned in, and defined by, the arm(s) 42 of the connector 22.

It is within the scope of the invention for the at least one hole 26 to be a blind hole or well 26 (i.e. a hole that does not extend between surfaces of the strut) or a through hole 26 (i.e. a hole that extends between the outer/abluminal surface and the inner/ luminal surface). Each hole 26 in a connector 22 can have the same shape, or a connector 22 can define holes 26 having different shapes. It is within the scope of the invention for a hole 26 to be any desired shape, for example, but not limited to round shaped, oval shaped, rectangular shaped, square shaped, and any combination thereof. A hole 26 can have a taper, with one end of the hole 26 having a greater dimension, such as diameter, than the other end of the hole 26. Alternatively the hole 26 can have no taper. Thus, the number and shape(s) of the holes 26 in FIGS. 3-29 are merely exemplary.

One of ordinary skill in the art will recognize that many different connectors 22 configurations can be designed from the combinations of number of arms 42 and body(ies) 40 as well as the different combinations of attributes that the arm(s) and body(ies), e.g. length, width, configurations, and holes can have.

As can be seen in the figures a hole 26 is different than a cell 28. A cell 28 is defined by a plurality of struts 12 and two connectors 22. A cell 28 that is a closed cell 28 is shown, for example, in FIG. 1. A cell 28 that is an open cell 28 is shown, for example, in FIG. 4. In contrast a hole 26 is defined by one strut 12, or by one connector 22, or by a portion of a connector 22 and a portion of a circumferential band 20.

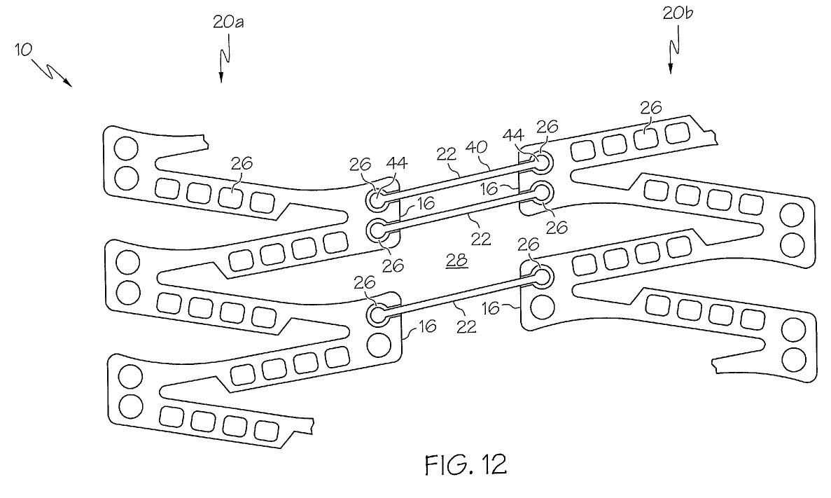

A connector 22 can engage adjacent circumferential bands 20 of struts 12 in different ways. Thus, the figures show only one possible attachment configuration of the connector embodiment 22 to the circumferential bands 20 and it is within the scope of the invention for the connector embodiment 22 have any type of attachment configuration. In at least one embodiment, the attachment configuration of the connector 22 affects the flexibility of the connector 22. In at least one embodiment, the attachment configuration of the connectors 22 affects the flexibility of the stent 10. In some embodiments, the same portions of adjacent circumferential bands 20 can be engaged by one or two connectors 22, shown, for example, in FIG. 12.

One attachment configuration is when a connector 22a engages the same sides of the struts 12a,b of adjacent circumferential bands 20a,b, as shown, for example, in FIG. 3. Note that both ends of the connector 22a are engaged to a mid-strut region 14. A mid-strut region 14 is the region of the strut 12 between the peak 16 and trough 18, as shown, for example, by the cross-hatching in FIG. 3. Note that sometimes, an end of a connector 22 can be positioned partly within a peak 16 and partly within a mid-strut region 14, as shown, for example, by the distal end of the connector 22 in FIG. 10, i.e. the end of the connector 22 that is engaged to the second circumferential band 20b.

Another attachment configuration is when the connector 22b engages opposite sides of struts 12c,d of adjacent circumferential bands 20a,b, as shown, for example, in FIG. 3. In this embodiment, both ends of the connector 22b are engaged to mid-strut regions 14 of the struts 12c,d. In other embodiments, the connector 22c,d engages a peak 16a,c of one circumferential band 20a and either a peak 16b, a peak to peak connector 22, or mid strut region 14 of a strut 12e of the adjacent circumferential band 20b, as shown, for example, in FIG. 3. Note that the peaks 16 to which the connector 22 is engaged can be longitudinally opposite one another, as shown for example by connector 22c in FIG. 3 or the peaks 16 can be circumferentially offset from one another, as shown, for example, in FIG. 4. In some embodiments, the connector 22 extends from a peak 16 of one circumferential band 20a to a trough 18 of the adjacent circumferential band 20b, a peak to trough connector 22, as shown, for example, in FIG. 6.

As discussed above, connectors 22 can have many different configurations or attributes. FIGS. 3-29 show non-limiting examples of different connectors 22 that have at least one of the attributes discussed herein. In FIG. 5, the connector 22 has one end engaged to a peak 16 of one circumferential band 20b and two second ends engaged to the adjacent circumferential band 20a at the mid strut regions 14 of adjacent struts 12. The connector 22 in FIG. 5 can also be described as having a body 40 and a V-shaped arm 42 engaged to one end of the body 40. As shown in FIG. 5, the connector 22 defines a hole 26 which is positioned at the junction of the body 40 of the connector 22 to the arm(s) 42.

In FIG. 6, the connector 22 is a peak 16 to trough 18 connector 22. The connector 22 has a body 40 with two holes 26 and two arms 42a,b. In this embodiment, the body 40 is round shaped and the arms 42a,b are substantially longitudinal and straight. As shown, one arm 42b has a greater length than the other arm 42a. In this embodiment, the holes 26 in the body 40 are a half-oval or half-round shape. Alternatively, the hole 26 can be described as being partially oval shaped or partially round shaped.

The connector in FIG. 7 has a body 40 and two Y-shaped arms 42a,b. The body 40 is oval shaped and has two holes 26b. The ends of each Y-shaped arm 42a,b are engaged to the peaks 16 of the adjacent circumferential bands 20a,b. In contrast to FIG. 6, the arms 42 of the connector 22 in FIG. 7 are approximately the same length. As shown in FIG. 7, a hole 26a,c is defined by a portion of the arm 42a,b of the connector 22 and a portion of circumferential band 20a,b. In this embodiment, the portion of the circumferential band 20a,b is a peak 16. However, if the Y-shaped arm was engaged to the strut 12 at the mid-strut region 14, the hole would be defined by a portion of the arm 42 and a portion of the strut 12.

In FIG. 8, the connector 22 has a zig-zag configuration and extends circumferentially peak 16a to peak 16b. The connector 22 in this embodiment has substantially the same width along the length of the connector 22. As shown in FIG. 8, the straight portions of the connector 22 define holes 26 but the turns 23 do not define any holes 26. However, in some embodiments, the turns 23 of the connector 22 define holes 26, as shown for example, in FIG. 3. As shown in FIG. 8, the connector 22 has the same number of holes 26 between turns 23. However, the number of holes 26 between turns 23 of the connector 22 can vary. Additionally the length between turns 23 can be the same of different.

In at least one embodiment, connectors 22 engaging adjacent circumferential bands 20a,b alternate between mid-strut 14 to peak attachment 22a and peak to mid-strut 14 attachment 22b, as shown, for example, in FIG. 9. As shown in FIG. 9, it is within the scope of the invention for the connectors 22 to extend from two adjacent strut 12 pairs on one circumferential band 20a to the same strut pair 12 on the adjacent circumferential band 20b.

FIG. 10 shows a connector 22 that has a zig-zag configuration. In this embodiment, some sections of the zig-zag are wider than other sections of the zig-zag. As used in this application, a section is a portion of the connector 22 between turns 23. It is within the scope of the invention for the sections of the zig-zag to be the same width or different widths. As shown in FIG. 10, the wider sections define larger holes 26a than the holes 26b of the narrower sections. In some embodiments, the zig-zag configuration of the connector 22 increases the flexibility of the connector 22. The connector 22 shown in FIG. 10 can also be described as having two bodies 40 and three arms 42. In this embodiment the bodies 40 define holes 26 and two of the arms 42 define holes 26.

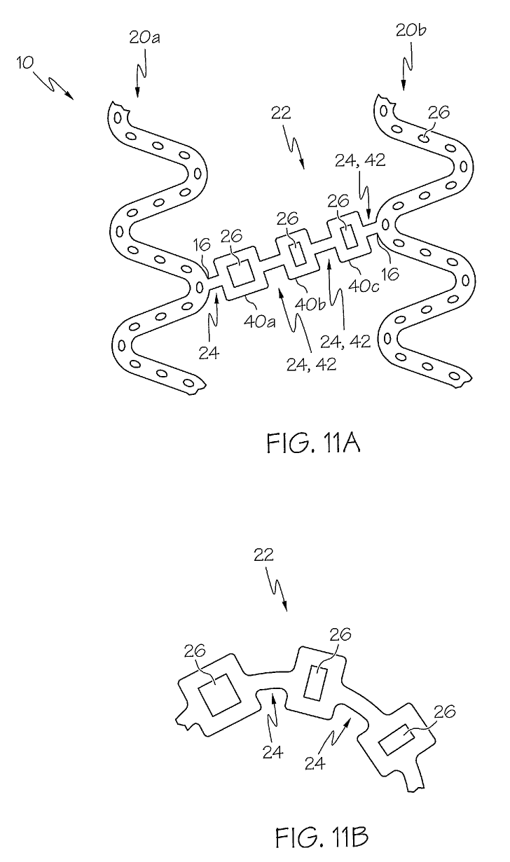

In at least one embodiment, the connector 22 has at least one flex point/zone of articulation 24, as shown, for example, in FIG. 11A. As used in this application, a flex point or zone of articulation 24 is an area that bends. In FIG. 11A, the connector 22 has four zones of articulation 24. In this embodiment, the zones of articulation 24 are straight when the connector 22 is in an unexpanded state and curved/bent when the connector 22 is in an expanded state, as shown, for example, in FIG. 11B. In some embodiments, the zones of articulation 24 are curved/bent when the connector 22 is in the unexpanded state and straight when the connector 22 is in an expanded state. A connector 22 can have any number of zones of articulation 24, including, but not limited to, one, two, three, four, five, six, seven, eight, nine, ten or more.

In some embodiments, the connector 22 is engaged to adjacent circumferential bands 20a,b by zones of articulation 24. In other embodiments, the portion of the connector 22 engaged to the circumferential bands 20a,b are not zones of articulation 24. Thus, in this embodiment the portion of the connector 22 engaged to the circumferential band 20 does not flex when the connector 22 is in the expanded state. In at least one embodiment, the connector 22 has at least one well/hole 26 between flex points/zones of articulation 24, as shown, for example, in FIG. 11A.

The connector 22 shown in FIG. 11A can also be described as having three bodies 40a,b,c and four arms 42 that are flex points/zones of articulation 24. Each body 40a,b,c defines one hole 26. One body 40a has a configuration that is square shaped while the other bodies 40b,c have a rectangular shaped configuration.

In at least one embodiment, the connector 22 has at least one knob 44 at at least one of the ends of the connector 22, as shown, for example, in FIG. 12. This type of connector 22 may be described as a knob connector 22. The knob connector 22 in FIG. 12 has two knobs 44 that have a shape that is complementary to the hole 26 in the peak 16 of the circumferential band 20. Thus, the knob 44 can have any configuration/shape so long as it is complementary to the configuration/shape of the hole 26. In FIG. 12, the knob 44 and the hole 26 each have a round configuration/shape. In some embodiments, the knob connector 22 has a body 40 and at least two arms 42 where one end of an arm 42 is engaged to the body 40 and the other end of an arm 42 is a knob 44 that is engaged to a circumferential band 20, as shown, for example, in FIG. 13. Also shown in FIG. 13, the body 40 is wider than the arms 42. In at least one embodiment, the body 40 defines at least one hole/well 26 to deliver a therapeutic agent. In FIG. 13 the arms 42 are engaged to the proximal and distal sides of the body 40, however, the arms 42 can be engaged to any portion of the body 40.

In some embodiments, the knob connector 22 is anchored by tension. In other embodiments, the knob connector 22 is anchored by an interference fit. In at least one embodiment, the knob connector 22 is press-fitted or insert molded. In at least one embodiment, a fixative or holding agent is added to the knob connector 22 before or after placement to engage the knob connector 22 to the circumferential bands 20. In some embodiments, the knob connector 22 is made from an inert material, for example, but not limited to, polytetrafluoroethylene (PTFE), polyvinylidene difluoride (PVDF), or polystyrene-polyisobutylene-polystyrene triblock copolymer (SIBS). Other example of materials that can be used for the connector 22 are discussed in greater detail below. In other embodiments, the knob connector 22 is impregnated with a therapeutic agent.

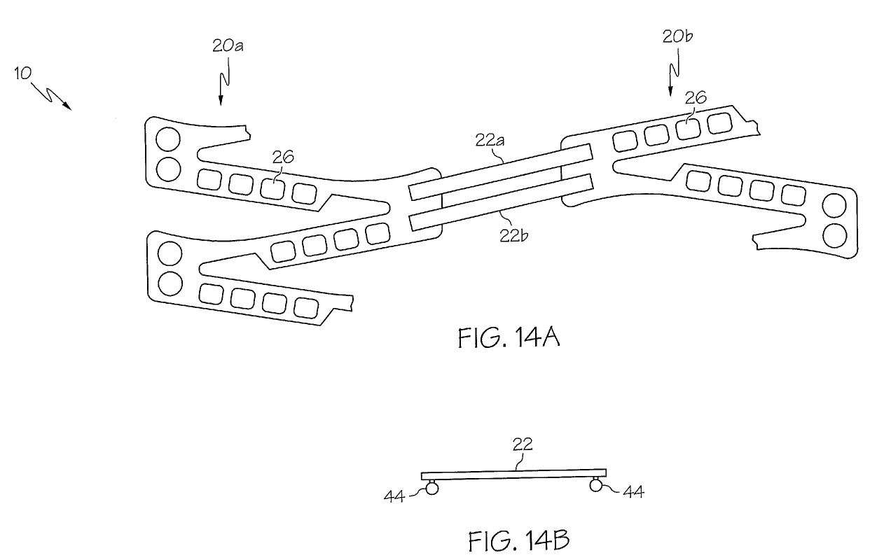

In at least one embodiment, a connector 22 with at least one knob is press fitted into holes 26 in adjacent circumferential bands 20a,b like a snap, as shown, for example, in FIGS. 14A and 14B. This type of connector 22 can be described as a snap connector 22. Thus, in this embodiment, the end regions of the connector 22 has a knob 44 that fits into the hole 26 in the circumferential band 20 so that the connector 22 snaps onto the stent 10. Note that the knob(s) of the snap connector 22 shown in FIGS. 14A and 14B is engaged to a side of the snap connector 22 while the knob(s) of the knob connector 22 shown in FIGS. 12-13 form an end of the knob connector 22. In at least one embodiment, the snap connector 22 is made of metal. In some embodiments, the snap connector 22 is made of Nitinol. It is also within the scope of the invention for the snap connector 22 to be made of a polymer, or a mixture of polymers. Other materials than can be used to form the snap connector 22 are discussed in greater detail below. In at least one embodiment, the snap connector 22 elutes a therapeutic agent.

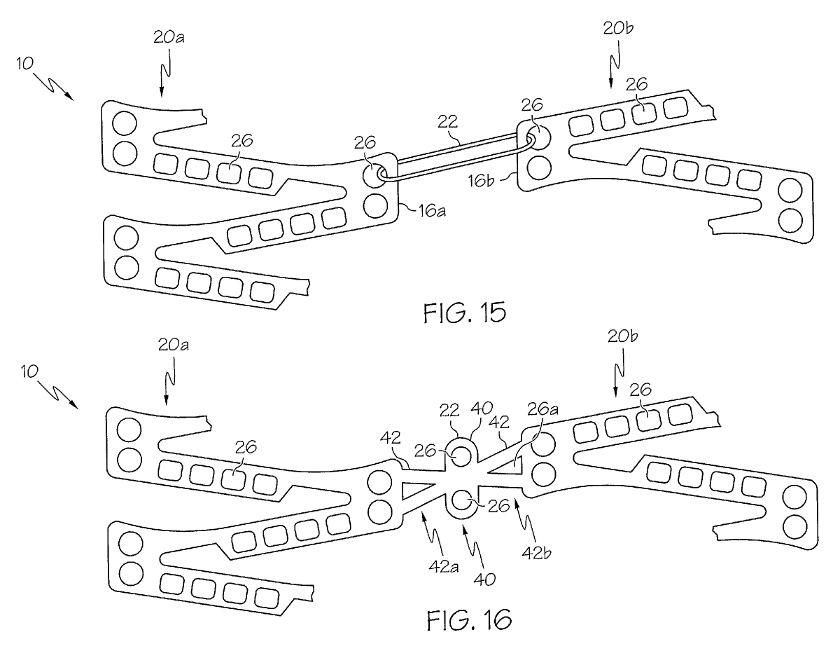

In at least one embodiment, the connector 22 engages adjacent circumferential bands 20a,b by extending through at least one hole 26 on one circumferential band 20a and at least one hole 26 on an adjacent circumferential band 20b, as shown, for example, in FIG. 15. It is within the scope of the invention for the configuration of the connector 22 to be in the form of a suture, a clip or a shoelace. In FIG. 15 the connector 22 is threaded through one of the holes 26 on the peak 16a of one circumferential band 20a and though one of the holes 26 on the peak 16b on the adjacent circumferential band 20b. Note that the connector 22 can be threaded through more than one hole 26 in a circumferential band 20. For example, the connector 22 can be arranged like a shoelace lacing up a shoe (not shown). In some embodiments, a therapeutic agent is deposited into the hole 26 after the connector 22 has been threaded through the hole 26.

The connector 22 in FIG. 16 comprises a body 40 with two holes 26 and two arms 42. In this embodiment, the body 40 has an oval shaped configuration and both arms 42 have a V-shape configuration. In at least one embodiment, the hole 26a defined by a V-shaped arm 42 and a portion of the circumferential band 20b has a therapeutic agent deposited therein.

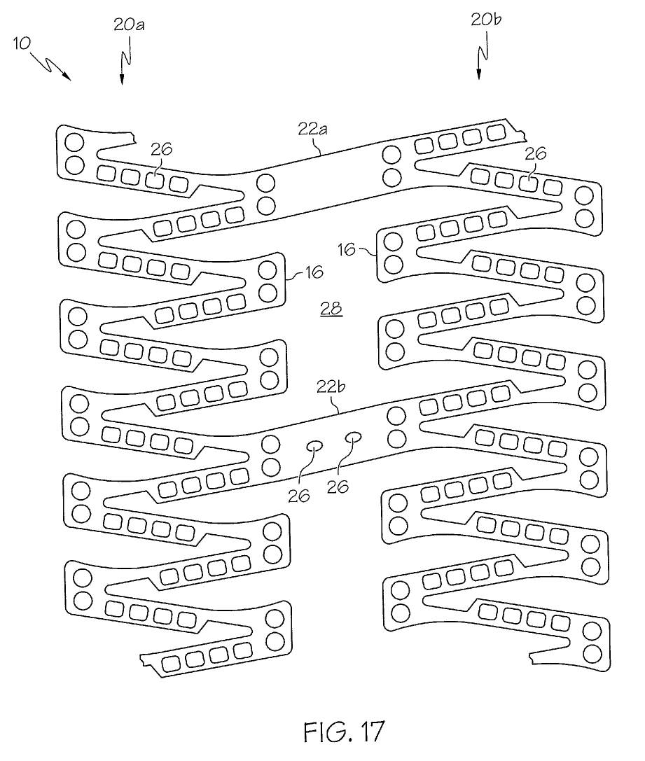

In at least one embodiment, the connector 22 is in the form of peaks 16 from adjacent circumferential bands 20a,b that are elongated so that they are engaged, as shown, for example, in FIG. 17. The elongated peak connector 22 can define at least one hole 26, as illustrated by connector 22b or the elongated peak connector 22 does not define any holes, as illustrated by connector 22a. In the embodiment shown in FIG. 17, the connector 22 has a width equal to the width of the peaks 16, but it is within the scope of the invention for the width of the connector to be smaller/narrower than at least one of the peaks 16, or larger/wider than at least one of the peaks 16.

The connector 22 in FIG. 18 can be described as having a body 40 and two 0-shaped arms 42. The body 40 has two holes 26 into which a therapeutic agent can be deposited. In some embodiments, a therapeutic agent is deposited in the hole 26a defined by the O-shaped arms 42. In at least one embodiment, the connector 22 has one to four zones of articulation 24. As shown in FIG. 18, each arm 42a,b has two zones of articulation 24. Alternatively, this connector 22 can be described as having three bodies, each body defining at least one hole 26 and engaged one to another by arms that are zones of articulation 24 and the connector 22 is engaged to each circumferential band 20 by an arm that is a zone of articulation 24.

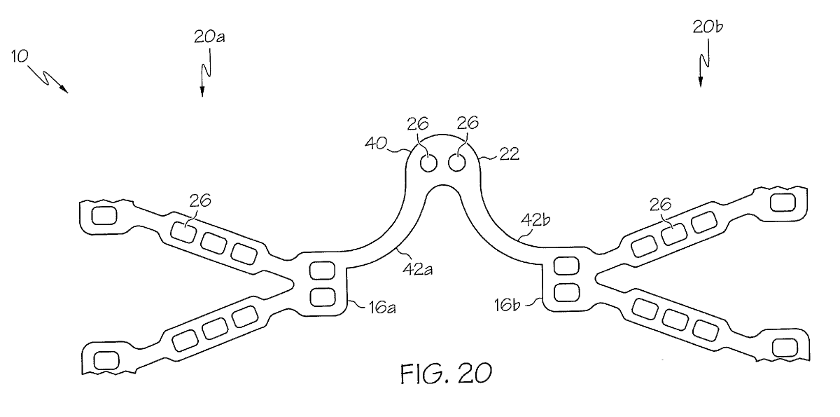

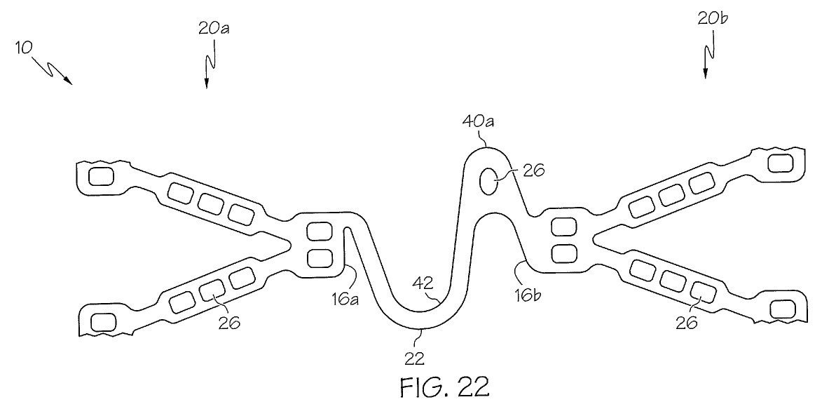

FIGS. 19-24 show different connector embodiments 22 that illustrate that connectors 22 can have first and second ends of different widths; that the ends of the connector 22 can have a width that is equal to or less than the width of a peak 16; that the connectors 22 can engage different portions of the peaks 16 of adjacent circumferential bands 20; that the connector 22 may or may not define holes 26; and that the position of the holes 26 defined by the connector 22 can vary. In FIGS. 19 and 22, the connector 22 has a first end that has a width equal to the width of a peak 16 of one circumferential band 20 and a second end that has a width less than the width of a peak 16b of the adjacent circumferential band 20. In FIGS. 20-21 and 23-24 the ends of the connector 22 have a width less than the widths of the peaks 16. Thus, the ends of the connector 22 are narrower than the peaks 16. The connector 22 in FIG. 19 does not define a hole 26 while the connectors 22 in FIGS. 20-24 each define at least one hole 26.

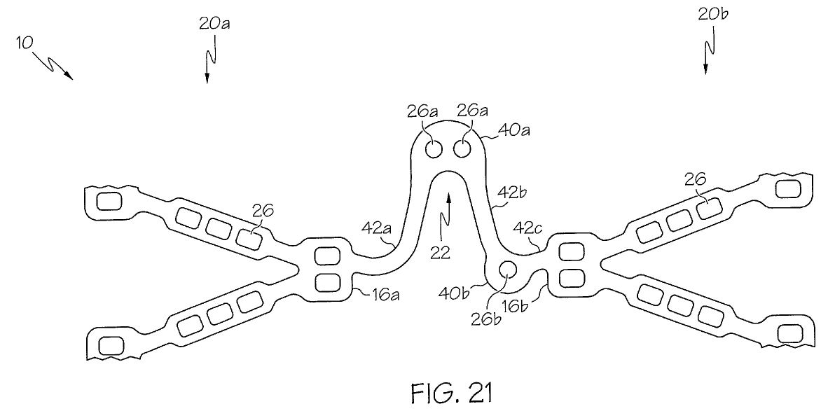

In FIG. 20, the connector 22 defines two holes 26 in the body 40 of the connector 22. The body 40 is positioned substantially halfway between the peaks 16a,b of the circumferential bands 20a,b. The connector 22 in FIG. 21 is similar to the connector 22 in FIG. 20 except that it has a second body 40b that defines one hole 26b. The second body 40b is positioned closer to the peak 16b of the second circumferential band 20b than the first body 40a, which is positioned substantially halfway between the peaks 16a,b of the circumferential bands 20a,b. Another difference of the connector 22 in FIG. 21 to the connector 22 in FIG. 20 is that the connector 22 in FIG. 21 has three arms 42a,b,c while the connector 22 in FIG. 20 has two arms 42a,b. The difference in the number of arms 42 in these two connectors 22 is due to the addition of a second body 40b to the connector 22 in FIG. 21. Another difference between the connectors 22 in FIGS. 19 and 20 are where the ends of the connectors 22 engage the peaks 16a,b of the circumferential bands 20a,b.

The connector 22 in FIG. 22 has a body 40a that defines one hole 26. The body 40a is positioned closer to one peak 16b than the other peak 16a. The body 40a is engaged to the first peak 16a of the first circumferential band 20a by an arm 42. The width of arm 42 is less than the width of the first peak 16a. Thus, the arm 42 is narrower than the peak 16a. The body 40a is also engaged to the peak 16b of the second circumferential band 20b but the connector 22 tapers from the body 40a to the peak 16b so that there is no clearly differentiated arm engaging the body 40a to the peak 16b.

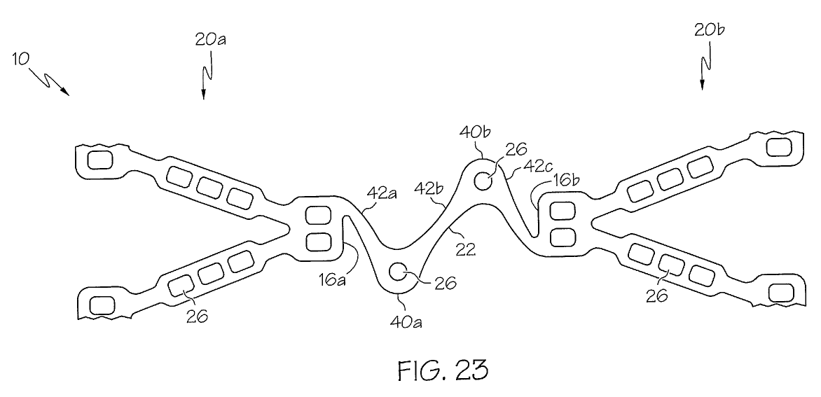

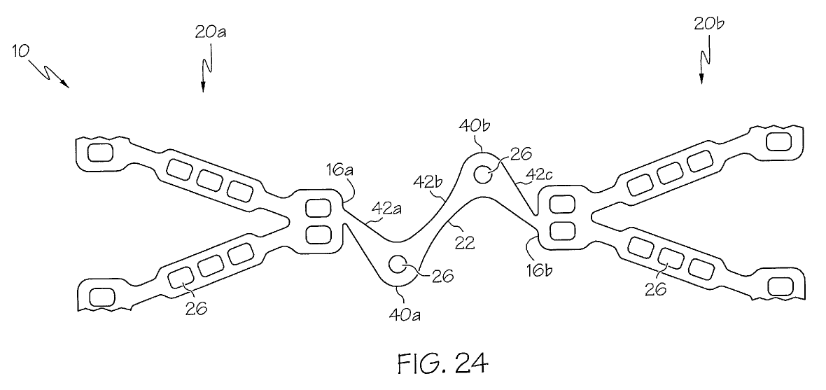

The connectors 22 in FIGS. 23 and 24 are similar. Both connectors 22 have two bodies 40a,b with each body 40a,b defining one hole 26. However, the first and third arms 42a,c of the connectors 22 are different. The first arm 42a of FIG. 23 is curvilinear and has a tapered end region where the arm 42a engages the peak 16a whereas the first arm 42a of FIG. 24 is straight and does not have a tapered end region. The arms 42a also are engaged to slightly different portions of the peak 16a. Additionally, the third arm 42c of FIG. 23 is curvilinear while the third arm 42c of FIG. 24 is straight and the third arms 42c of FIGS. 23 and 24 are engaged to different portions of the peak 16b.

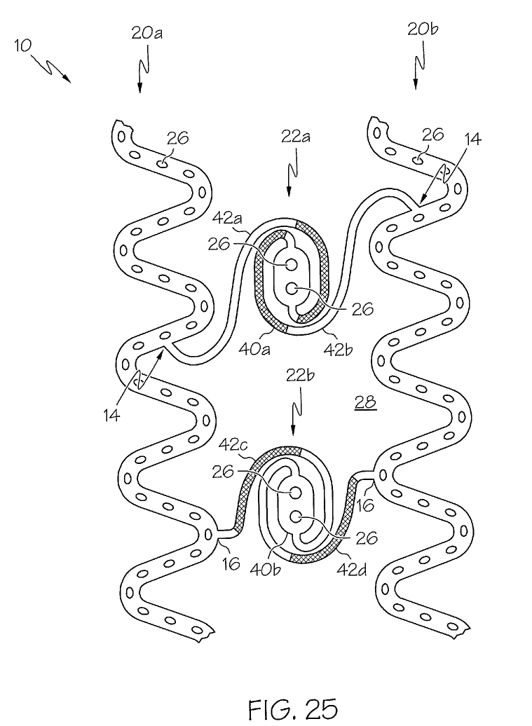

Another connector embodiment 22 is illustrated in FIG. 25. Both connectors 22a,b in FIG. 25 have a body 40a,b that is oval shaped and which defines two holes 26. The arms 42a,b of the first connector 22a are engaged to the mid-strut regions 14 of the struts 12 of adjacent circumferential bands 20a,b while the arms 42c,d of the second connector 22b are engaged to the peaks 16 of the adjacent circumferential bands 20a,b. In this embodiment, a portion of each arm 42a,b extends about/around the body 40a,b so that the arm 42a,b is adjacent to the body 40a,b, shown, for example, by the cross-hatching of connector 22a, and a portion of each arm 42a,b extends about/around a portion of the other arm 42a,b so that at least a portion of the arms 42a,b are adjacent to one another, as indicated by cross-hatching.

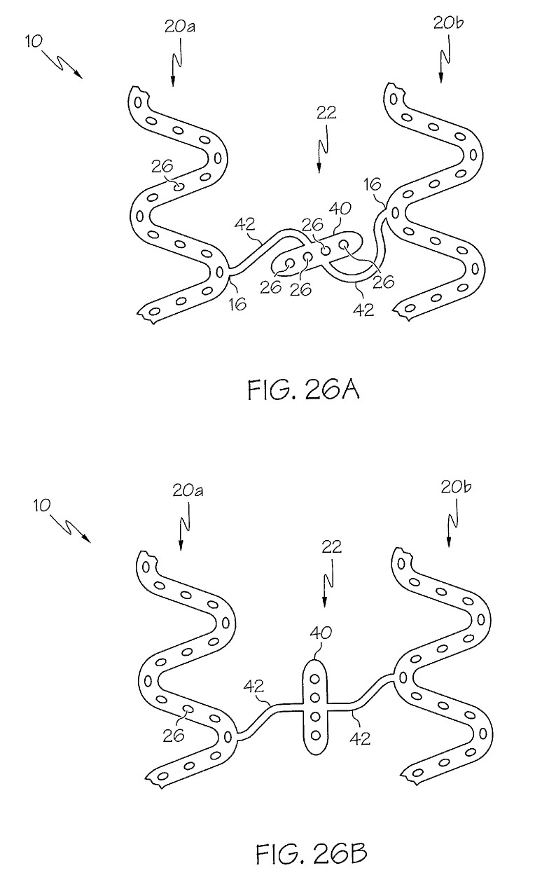

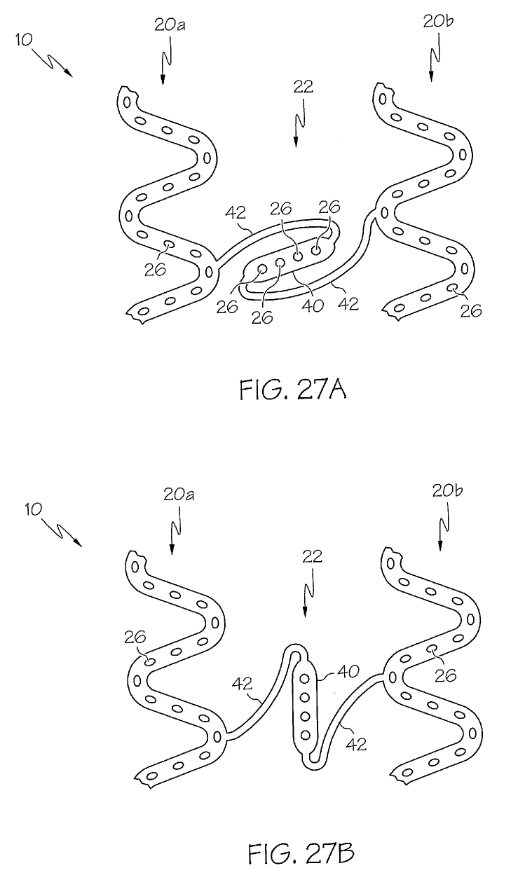

FIGS. 26-27 show two variations of another connector embodiment 22. As shown in FIG. 26A, the connector 22 has a body 40 and two arms 42. The body 40 is oval shaped and defines four holes 26. The arms 42 extend around at least a quarter of the body 40 of the connector 22. The arms 42 are engaged to the sides of the body 40 and to the peaks of the adjacent circumferential bands 20a,b. In contrast, the arms 42 of the connector 22 in FIG. 27A are engaged to the ends of the body 40. The connector 22 has a pre-expansion state, shown in FIGS. 26A and 27A and a post-expansion state, shown in FIGS. 26B and 27B. The connector 22 is in the pre-expansion state when the stent 10 is in an unexpanded state. Similarly, the connector 22 is in the post-expansion state when the stent 10 is in a deployed or expanded state.

In at least one embodiment, the orientation of the body 40 of the connector 22 relative to the longitudinal axis of the stent 10 changes from the pre-expansion state to the post-expansion state. In some embodiments, the orientation of the body 40 of the connector 22 relative to the longitudinal axis of the stent 10 changes from a first oblique angle in the pre-expansion state to a second oblique angle to the longitudinal axis in the post-expansion state. As used in this application, an oblique angle is an angle between 0 and 180 and includes 90 degrees, where 0/180 is the longitudinal axis of the stent 10. In other embodiments, the orientation of the body 40 of the connector 22 relative to the longitudinal axis of the stent 10 changes from being parallel to the longitudinal axis of the stent 10 in the pre-expansion state to an oblique angle in the post-expansion state. In some embodiment, the body 40 of the connector 22 is perpendicular to the longitudinal axis of the stent 10 when the connector is in the post-expansion state. In at least one embodiment, the orientation of the body 40 of the connector 22 in the post-expansion state increases the amount of scaffolding between adjacent circumferential bands 20.

FIG. 28 shows a stent 10 with four circumferential bands 20 engaged by connectors 22 which are arranged in three different alternative ways 22a,b,c. Although the connectors 22 are engaged to the mid-strut region 14 of the struts 12, these non-limiting examples of connector alignment can also be achieved with connectors 22 engaged peak to peak, peak to trough, or trough to trough, peak to mid-strut region, trough to mid-strut region, mid-strut region to mid-strut region, and any combination thereof.

In at least one embodiment, the connectors 22 of the stent 10 can be aligned longitudinally, as shown by the connectors 22a and 22b in FIG. 28. With the connector alignment represented by 22a, the mid-strut regions 14 to which the connectors 22 are engaged are aligned longitudinally and the connectors 22 are longitudinally oriented. Note that the first and second ends of each connector 22 have substantially the same position about the circumference of the stent 10 (same circumferential position). In contrast, with the connector alignment represented by 22b, the connectors 22 are circumferentially oriented, with longitudinally adjacent connectors 22 being oriented at different oblique angles to the longitudinal axis of the stent 10. Note that the first and second ends of each connector 22 are circumferentially offset (one end of the connector 22 has a different circumferential position than the other end).

In at least one embodiment, the connectors 22 of the stent 10 are aligned at an oblique angle to the longitudinal axis of the stent 10, as shown for example by the connectors 22c in FIG. 28. In some embodiments, the connectors 22 form a helical pathway about the stent 10. Again, the first and second end of each connector 22 are circumferentially offset.

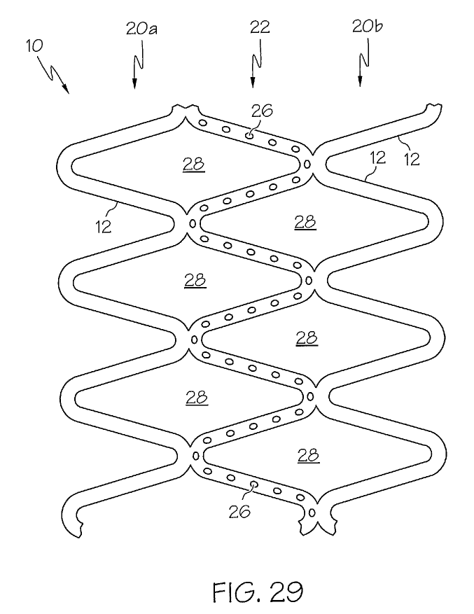

In at least one embodiment, the connector 22 is a circumferential band that engages adjacent circumferential bands 20a,b of struts 12, as shown for example in FIG. 29. In some embodiments, both the connector 22 and the circumferential bands 20 of struts 12 have holes 26. In other embodiments, only the connector 22 has holes 26.

The circumferential bands 20 and connectors 22 of the stent 10 may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The circumferential bands 20 and connectors 22 of the stent 10 may be made of shape memory materials such as superelastic Nitinol or spring steel, or may be made of materials which are plastically deformable. In the case of shape memory materials, the circumferential bands 20 and connectors 22 of the stent 10 may be provided with a memorized shape and then deformed to a reduced diameter shape. The circumferential bands 20 and connectors 22 of the stent 10 may restore itself to its memorized shape upon being heated to a transition temperature and having any restraints removed therefrom.

The circumferential bands 20 and connectors 22 of the stent 10 may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent 10, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque. In at least one embodiment, the stent 10 has at least one feature designed to be resonant at imaging frequencies or to preferentially absorb specific frequencies to create a marker or transducer.

In some embodiments the at least a portion of the stent 10 is configured to include one or more mechanisms for the delivery of a therapeutic agent. Although the elution of a therapeutic agent from area(s) of a connector 22 may have been discussed with regard to a specific embodiment of a connector 22, it is within the scope of the invention for a therapeutic agent to be eluted from any type of connector 22. Often the agent will be in the form of a coating or other layer (or layers) of material placed on a surface region of the stent, which is adapted to be released at the site of the stent's implantation or areas adjacent thereto. In at least one embodiment, at least one therapeutic agent is deposited within and eluted from the holes 26. In some embodiments, at least a portion of the material(s) forming the stent 10 is impregnated with at least one therapeutic agent.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. An expandable stent, the stent comprising a plurality of expandable rings formed of a plurality of struts and at least one first connector interconnecting adjacent expandable rings, the plurality of struts comprising a first strut, the at least one first connector comprising a first arm, a first portion of the first arm engaged to a first portion of the first strut so that the first portion of the first arm and the first portion of the first strut define a through hole.

2. The expandable stent of claim 1, the first portion of the first arm having a configuration selected from at least one member of the group consisting of Y-shaped, V-shaped, U-shaped and X-shaped.

3. The expandable stent of claim 2, the first arm having a second portion, the second portion of the first arm being engaged to the first portion of the first arm, the second portion having a configuration selected from at least one member of the group consisting of zig-zag, straight, curvilinear, O-shaped, V-shaped, U-shaped, X-shaped, Y-shaped and any combination thereof.

4. The expandable stent of claim 1, the at least one first connector defining at least one hole.

5. The expandable stent of claim 2, the first arm having a first width, the at least one first connector further comprising a body, the body having a second width, the second width being at least equal to the first width, the body engaged to the first arm at a junction area.

6. The expandable stent of claim 5, the at least one first connector defining at least one hole, the at least one hole positioned at the junction area.

7. The expandable stent of claim 1, the plurality of struts comprising a second strut, the first strut forming a portion of a first expandable ring, the second strut forming a portion of a second expandable ring, the first expandable ring adjacent to the second expandable ring, the at least one first connector engaging the first and second expandable rings, the at least one first connector further comprising a second arm, a first portion of the second arm being engaged to a first portion of the second strut so that the first portion of the second arm and the first portion of the second strut define a through hole.

8. The expandable stent of claim 7, the first arm having a first width, the second arm having a second width, the at least one first connector further comprising a body, the body having a third width, the third width being greater than the first width, the third width being greater than the second width, the body engaging the first and second arms.

9. The expandable stent of claim 8, the body defining at least one hole.

10. The expandable stent of claim 8, the first portion of the first arm having a configuration selected from at least one member of the group consisting of Y-shaped, V-shaped, U-shaped and X-shaped and the first portion of the second arm having a configuration selected from at least one member of the group consisting of Y-shaped, V-shaped, U-shaped and X-shaped.

11. An expandable stent, the stent comprising a plurality of expandable rings formed of a plurality of struts and at least one first connector interconnecting adjacent expandable rings, the at least one first connector comprising a body, a first arm and a second arm, the body defining at least two through holes, a first portion of the first arm extending around a first portion of the body, a second portion of the first arm extending about a first portion of the second arm, the first portion of the second arm extending around a second portion of the body, a second portion of the second arm further extending about a second portion of the first arm, the first and second portions of the first arm being different portions of the first arm and the first and second portions of the second arm being different portions of the second arm.

12. An expandable stent having a longitudinal axis, the stent comprising a plurality of expandable rings formed of a plurality of struts and at least one first connector interconnecting adjacent expandable rings, the at least one first connector comprising a body, a first arm and a second arm, the body of the at least one first connector having a first position when the stent is in an unexpanded state and a second position when the stent is in an expanded state, the first position being at a first angle to the longitudinal axis of the stent and the second position being at a second angle to the longitudinal axis of the stent, the first angle being different than the second angle.

13. The stent of claim 12, the body comprising at least one mechanism to deliver a therapeutic agent.

14. The stent of claim 13, wherein the at least one mechanism to deliver a therapeutic agent is a through hole.

15. An expandable stent, the stent comprising a plurality of expandable rings formed of a plurality of struts and at least one first connector interconnecting adjacent expandable rings, the plurality of struts comprising a first strut and a second strut, the first strut forming a portion of a first expandable ring, the second strut forming a portion of a second expandable ring, the first and second expandable rings being adjacent, the first strut defining a first hole, the second strut defining a second hole, the at least one first connector comprising a body, a first arm with a first knob and a second arm with a second knob, the at least one first connector interlocking the first and second struts by the first knob being interlocked with the first hole of the first strut and the second knob being interlocked with the second hole of the second strut.

16. The stent of claim 15, the at least one first connector having a first side, the first and second knobs being engaged to the first side.

17. The stent of claim 15, the first knob forming an end of the first arm and the second knob forming an end of the second arm.

18. The stent of claim 15, the body having a first width, the first arm having a second width, the second arm having a width, the first, second and third widths being the same.

19. The stent of claim 15, the body having a first width, the first arm having a second width, the second arm having a width, the first width being different than the second and third widths.

20. The stent of claim 15, the body having a first configuration, the first arm having a second configuration, the second arm having a third configuration, the first configuration being different than the second and third configurations.
